(19) United States
(12) Patent Application Publication
(10) Pub. No.: US 2006/0135906 A1
(43) Pub. Date: Jun. 22, 2006

(54) Iontophoretic device and method for administering immune response-enhancing agents and compositions

(76) Inventors: Akihiko Matsumura, Shibuya-ku (JP); Steven G. Reed, Bellevue, WA (US)

Correspondence Address: SEED INTELLECTUAL PROPERTY LAW GROUP PLLC, 701 FIFTH AVE, SUITE 6300, SEATTLE, WA, 98104-7092, US

(21) Appl. No.: 11/280,805

(22) Filed: Nov. 16, 2005

Related U.S. Application Data
(60) Provisional application No. 60/627,952, filed on Nov. 16, 2004.
(60) Provisional application No. 60/714,538, filed on May 16, 2005.

(51) Int. Cl. A61N 1/30; A61K 39/02; A61K 31/739

(52) U.S. Cl. 604/20; 424/234.1; 514/54

(57) ABSTRACT

An iontophoresis device and method delivers an immune response-enhancing agent, or composition thereof via iontophoresis. The device may include an active electrode assembly having a drug solution holding portion, comprising an immune response-enhancing agent, or composition thereof; and a non-active electrode assembly. An iontophoresis device delivers an immune response-enhancing agent via iontophoresis. The device includes an active electrode assembly and a non-active electrode assembly, wherein the active electrode assembly comprises: a first electrode element operable to provide an electrical potential of a first polarity and a drug solution holding portion arranged on a front surface of the first electrode member.

### CROSS-REFERENCE TO RELATED APPLICATION

This application claims the benefit under 35 U.S.C. § 119(e) of U.S. Provisional Patent Application No. 60/627,952 filed Nov. 16, 2004; and U.S. Provisional Patent Application No. ______, converted (Express Mail EV 718205539US) from U.S. Non-Provisional patent application Ser. No. 11/129,321, filed May 16, 2005, where these two provisional applications are incorporated herein by reference in their entireties.

### BACKGROUND OF THE INVENTION

1. Field of the Invention

This disclosure generally relates to methods and devices for administering immune response-enhancing agents or compositions. The disclosure relates more particularly to methods for administering adjuvants and adjuvant-containing compositions and to iontophoretic devices suitable for administration of such agents and compositions.

2. Description of the Related Art

Methods currently used for delivering compositions for enhancing or stimulating an immune response, such as immunization against infectious diseases, generally require penetration of skin or mucous membrane, for example by use of a needle. Such methods are performed under sterile conditions and require trained personnel. Such conditions and personnel are not always readily available. Furthermore, repeat use of needles under non-sterile conditions can lead to transfer of disease. In addition, due to pain, as well as risk of infection, many individuals hesitate to comply with treatment regimens, particularly when repeat administration is required for treatment or prophylaxis. Furthermore, when medicines are to be administered to infants who have a thin skin or to small animals, particularly skilled personnel are necessary. Development of methods for needle-free administration of immune response-enhancing or stimulating agents or compositions is thus a priority.

U.S. Pat. No. 6,797,276 describes a system for passive transcutaneous immunization, wherein delivery of antigen is targeted to the Langerhans islet cells, located in the outermost layer of skin. U.S. Pat. No. 5,910,306 describes application of formulations comprising antigen and liposomes to skin, while U.S. Pat. No. 5,980,898 describes a patch for passive transcutaneous immunization comprising an antigen, an adjuvant and a dressing. Each of these patents is incorporated herein by reference in their entirety.

A known administration method to overcome the various problems associated with injection is iontophoresis (also termed as “iontophorese”, “ion-introducing method”, or “ion penetration therapy”). Iontophoresis is used for transdermal delivery of a drug or active agent, typically ionic or polar, into the body through the skin or mucosa by application of an electromotive force sufficient to drive, or carry, the drug or active agent into or through the skin or mucosa. In this delivery method, for example, a positively charged drug may be driven into or through the skin by the force applied from an anode, or a negatively charged drug may be driven into or through the skin by the force applied from a cathode. During iontophoresis, in addition to migration of charged molecules in response to repulsive forces, charged or uncharged drugs or active agents may also be carried into or through the skin by electroosmotic solvent flow.

Investigation of the use of iontophoresis for delivery of drugs or active agents through the skin or mucous membranes has typically been described for the delivery of small ionic or polar drugs or active agents. Examples of such drugs or active agents to which iontophoresis may be applied as a method for delivery include anesthetics such as procaine hydrochloride and lidocaine; gastrointestinal disease remedies such as carnitine hydrochloride; skeletal muscle relaxants such as vancronium bromide; antibiotics such as tetracycline based preparations, kanamycin based preparations, and gentamycin based preparations; vitamins such as B-2, B-12, C, E, and folic acid; adrenocortical hormones such as hydrocortisone based water-soluble preparations, dexamethasone based water-soluble preparations, and prednisolone based water-soluble preparations; antibiotics such as penicillin based water-soluble preparations and chloramphenicol based water-soluble preparations.

Iontophoresis has not typically been described for the delivery of larger drugs or active agents or those that are nonionic and have limited solubility in aqueous media. For example, immune response-enhancing adjuvants, such as lipid A and lipid A analogues, which have low water solubility and molecular weights greater than 1000, do not appear to have been studied as objectives for iontophoretic transcutaneous delivery.

The approaches described herein are intended to address some of the above-mentioned problems by using an iontophoresis device that includes a plurality of ion-exchange membranes. Various types of apparatus for administering drugs by iontophoresis have been known.

A discussion and examples of iontophoresis devices having ion-exchange membranes follows.

JP 03-504343 A discloses an iontophoresis electrode that includes (i) an electrode section, (ii) a reservoir that contains an ionic or ionizable medicine to be penetrated, and (iii) an ion-exchange membrane that is provided outside the reservoir (on the side that contacts the skin) and that selects the same ion as the charged ion of the ionic medicine. The ion-exchange membrane is described, for example, as limiting the migration of ion species, such as sodium and chloride, from the skin into the drug-containing electrode assembly.

U.S. Pat. No. 4,722,726 B discloses an electrode that includes (i) an upper chamber filled with a buffer solution and (ii) a lower chamber filled with an anionic drug, separated from the upper chamber by an ion-exchange membrane, the purpose being to mitigate adverse effects due to hydrolysis of water.

JP 03-94771 A discloses an iontophoresis electrode that includes (i) a moisture holding section surrounded by a resilient supporting member and having an electrode plate therein, (ii) an ion-exchange membrane arranged in front of the moisture holding section (on the side of the skin), and (iii) a drug layer (ionic drug layer) arranged in front of the ion-exchange membrane (on the side of the skin). The drug is spray dried or adhered or attached to the surface of the ion-exchange membrane that contacts the skin.

Adjuvants generally are agents that are used to enhance the effectiveness of, for example, a pharmacological compound. In particular, adjuvants are administered with vaccines or antigens to enhance the immune response to the vaccine or antigen. Adjuvants are effective when delivered to the epidermis in which Langerhans islet cells are present. Therefore, adjuvants, such as lipid A or lipid A analogues, are typically administered by injection into the epidermis.

Lipid A is an active center of lipopolysaccharides (LPS) obtained from gram-negative bacteria. Lipid A has an interferon inducing effect and a TNF inducing effect. In addition, lipid A has immunostimulating effects such as a macrophage activating effect, a β-cell juvenizing effect, and a cellular immunostimulating effect. Utilization of Lipid A as an adjuvant to be administered together with various vaccines is being studied. Some lipid A derivatives maintain or increase the immunostimulating effect of Lipid A as described above while they have eliminated toxicity or harmful effects. Such lipid A derivatives have a disaccharide structure (4-O-2-amino-2-deoxy-β-D-glucopyranosyl-amino-2-deoxy-D-glucopyranose) that consists of two D-glucosamine molecules connected through a β1-6 bond as a basic skeleton. A lot of compounds including monophosphoryl lipid A, 3-O-deacylated monophosphoryl lipid A, aminoalkylglucosaminide 4-phosphates (AGP) and so on (hereafter, referred to as “lipid A analogues” in the present specification) have been synthesized as the lipid A derivatives (see David et al., “Lipid A Analogues as Adjuvant and Immunoactivator”, 2002, Trend in Microbiology, Vol. 10, No. 10, page S32, Baker et al., “Inactivation of Suppressing T Cell Activity by Nontoxic Monophosphoryl Lipid A”, Interaction and Immunity, 1998, Vol. 56, No. 5, page 1076, and U.S. Pat. No. 4,912,094 B).

Iontophoresis devices, including those disclosed in JP 03-504343 A, U.S. Pat. No. 4,722,726 B, and JP 03-94771 A, do not appear to have been used to successfully administer lipid A or lipid A analogues into the epidermis in amounts sufficient to generate immunologically significant immune response-enhancing effects.

Related matters, as described in David et al., “Lipid A Analogues as Adjuvant and Immunoactivator”, 2002, Trend in Microbiology, Vol. 10, No. 10, page S32; Baker et al., “Inactivation of Suppressing T Cell Activity by Nontoxic Monophosphoryl Lipid A”, Interaction and Immunity, 1998, Vol. 56, No. 5, page 1076; U.S. Pat. No. 4,912,094 B; JP 03-504343 A; U.S. Pat. No. 4,722,726 B; JP 03-94771; JP 04-297277 A; JP 2000-229128 A; JP 2000-229129 A; JP 2000-237326 A; JP 2000-237327 A; JP 2000-237328 A; JP 2000-237329 A; JP 2000-288097 A; JP 2000-288098 A; JP 2004-188188 A; and WO 03/037425, are incorporated herein by reference as far as consistent to the disclosure herein.

Use or investigation of a variety of other adjuvants, in addition to lipid A and lipid A analogues, have been described or are under investigation for enhancing the immune response to various immune stimulants. Such adjuvants include saponin, such as QS-21, or derivatives thereof; CpG; imiquimod; resiquimod; dSLIM; and agonist of toll-like receptors, such as TLR-2, TLR-4, TLR-5, TLR-7, and TLR-9. Such adjuvants may enhance the immune response to a variety of vaccines, antigens and allergens.

In view of the various issues noted above related to the current methods for administration of immune response-stimulating or immune response-enhancing agents or drugs, there is a need in the art for improved devices and methods for the effective, safe, painless transcutaneous administration of such agents or drugs.

### BRIEF SUMMARY OF THE INVENTION

An iontophoresis device for administering an immune response-enhancing agent, or composition thereof, the iontophoresis device, comprising: an active electrode assembly having a drug solution holding portion, comprising an immune response-enhancing agent, or composition thereof, and a non-active electrode assembly.

In certain embodiments of the iontophoresis device, the immune response-enhancing agent is an adjuvant. In certain embodiments, the adjuvant may be lipid A or an analogue of lipid A. In certain such embodiments, the analogue of lipid A may be selected from monophosphoryl lipid A (MPL); 3-O-deacylated monophosphoryl lipid A; or aminoalkylglucosamine 4-phosphate. In certain other embodiments, the adjuvant may be an agonist of a toll-like receptor. In certain such embodiments, the toll-like receptor may be selected from TLR-2; TLR-4; TLR-5; TLR-7; or TLR-9. In yet other embodiments, the adjuvant is a saponin or a derivative thereof. In certain such embodiments, the saponin or derivative thereof is QS-21. In further embodiments, the adjuvant is selected from CpG; imiquimod; resiquimod; or dSLIM.

In certain embodiments, the drug solution holding portion of the iontophoresis device further comprises a vaccine or antigen. In certain embodiments, the vaccine or antigen comprises at least one antigen selected from viral antigens; bacterial antigens (including bacterial endotoxin); protozoal antigens; or parasite antigens. In certain such embodiments, the parasite antigen is selected from leishmania antigens or malaria antigens. In certain other embodiments, the vaccine or antigen comprises at least one antigen selected from hepatitis antigens (including hepatitis A, hepatitis B, or hepatitis C); hepatitis B surface antigen (HbsAg); mutants of hepatitis B surface antigen; and influenza antigens. In yet other embodiments, the vaccine or antigen comprises at least one antigen selected from Bordetella pertussis (pertussis) antigens; Corynebacterium diphtheriae (diphtheria) antigens; Chlostridium tetani (tetanus) antigens; influenza B viral antigens; or polio virus antigens. In further embodiments, the vaccine or antigen comprises an antigen mixture selected from mixtures of DTP (diphtheria, tetanus, pertussis) and HbsAg (hepatitis B surface antigen); mixtures of Hib (haemophilus influenzae type b) and HbsAg; mixtures of DTP, HbsAg, and Hib; or mixtures of IPV (inactivated polio vaccine), DTP, HbsAg, and Hib.

In certain embodiments, the drug solution holding portion of the iontbphoresis device further comprises a cancer antigen. In certain such embodiments, the cancer antigen is selected from melanoma antigens; basal cell carcinoma antigens; breast cancer antigens; prostate cancer antigens; lung cancer antigens; or ovarian cancer antigens.

In certain embodiments, the drug solution holding portion of the iontophoresis device comprises an allergen. In certain such embodiments, the allergen is selected from insect venoms; plant pollens; house dust mites; animal dander; ragweed; or endotoxin.

An iontophoresis device for administering an immune response-enhancing agent, or composition thereof, the iontophoresis device, comprising: an active electrode assembly having a drug solution holding portion, comprising an immune response-enhancing agent, or composition thereof, and a non-active electrode assembly; wherein the active electrode assembly further comprises: a first electrode member operable to provide an electrical potential of a first polarity; the drug solution holding portion arranged on the front surface of the electrode member; and a first ion-exchange membrane arranged on the front surface of the drug solution holding portion; and wherein the non-active electrode assembly comprises: a second electrode member operable to provide an electrical potential of a second polarity; and a first electrolyte solution holding portion arranged on the front surface of the second electrode member.

In certain embodiments of the iontophoresis device, the active electrode assembly of the device further comprises: a second electrolyte solution holding portion arranged on the front surface of the first electrode member; and a second ion-exchange membrane interposed between the second electrolyte solution holding portion and the drug solution holding portion. In certain other embodiments, the non-active electrode assembly of the device further comprises: a third ion-exchange membrane arranged on the front surface of the first electrolyte solution holding portion. In certain other embodiments, the non-active electrode assembly further comprises: a fourth ion-exchange membrane arranged on the front surface of the first electrolyte solution holding portion; and a third electrolyte solution holding portion interposed between the fourth ion-exchange membrane and the third ion-exchange membrane. In certain other embodiments, the first polarity is a negative polarity; the second polarity is a positive polarity; the first ion-exchange membrane and the fourth ion-exchange membrane are anion-exchange membranes; the second ion-exchange membrane and the third ion-exchange membrane are cation-exchange membranes; and the immune response-enhancing agent is lipid A or a lipid A analogue. In yet other embodiments, the lipid A analogue is selected from monophosphoryl lipid A (MPL); 3-O-deacylated monophosphoryl lipid A; and aminoalkylglucosaminide 4-phosphate.

A method for administering an immune response-enhancing agent, or composition thereof, using an iontophoresis device, the device, comprising: an active electrode assembly having a drug solution holding portion, comprising an immune response-enhancing agent, or composition thereof; and a non-active electrode assembly; the method comprising: electrically coupling the active electrode assembly and the non-active electrode assembly to poles of a power source; and applying a voltage or current to the active electrode assembly and the non-active electrode assembly; wherein the active electrode assembly and the non-active electrode assembly are brought into contact with a skin of a mammal.

In certain embodiments of the method for administering an immune response-enhancing agent, or composition thereof, using an iontophoresis device, the immune response-enhancing agent is an adjuvant. In certain embodiments of the method, the adjuvant may be lipid A or an analogue of lipid A. In certain such embodiments of the method, the analogue of lipid A may be selected from monophosphoryl lipid A (MPL); 3-O-deacylated monophosphoryl lipid A; or aminoalkylglucosamine 4-phosphate. In certain other embodiments of the method, the adjuvant may be an agonist of a toll-like receptor. In certain such embodiments of the method, the toll-like receptor may be selected from TLR-2; TLR-4; TLR-5; TLR-7; or TLR-9. In yet other embodiments of the method, the adjuvant is a saponin or a derivative thereof. In certain such embodiments of the method, the saponin or derivative thereof is QS-21. In further embodiments of the method, the adjuvant is selected from CpG; imiquimod; resiquimod; or dSLIM.

In certain embodiments of the method for administering an immune response-enhancing agent, or composition thereof, using an iontophoresis device, the drug solution holding portion of the device further comprises a vaccine or antigen. In certain embodiments of the method, the vaccine or antigen comprises at least one antigen selected from viral antigens; bacterial antigens (including bacterial endotoxin); protozoal antigens; or parasite antigens. In certain such embodiments of the method, the parasite antigen is selected from leishmania antigens or malaria antigens. In certain other embodiments of the method, the vaccine or antigen comprises at least one antigen selected from hepatitis antigens (including hepatitis A, hepatitis B, or hepatitis C); hepatitis B surface antigen (HbsAg); mutants of hepatitis B surface antigen; and influenza antigens. In yet other embodiments of the method, the vaccine or antigen comprises at least one antigen selected from Bordetella pertussis (pertussis) antigens; Corynebacterium diphtheriae (diphtheria) antigens; Chlostridium tetani (tetanus) antigens; influenza B viral antigens; or polio virus antigens. In further embodiments, the vaccine or antigen comprises an antigen mixture selected from mixtures of DTP (diphtheria, tetanus, pertussis) and HbsAg (hepatitis B surface antigen); mixtures of Hib (haemophilus influenzae type b) and HbsAg; mixtures of DTP, HbsAg, and Hib; or mixtures of IPV (inactivated polio vaccine), DTP, HbsAg, and Hib.

In certain embodiments of the method for administering an immune response-enhancing agent, or composition thereof, using an iontophoresis device, the drug solution holding portion of the device further comprises a cancer antigen. In certain such embodiments of the method, the cancer antigen is selected from melanoma antigens; basal cell carcinoma antigens; breast cancer antigens; prostate cancer antigens; lung cancer antigens; or ovarian cancer antigens.

In certain embodiments of the method for administering an immune response-enhancing agent, or composition thereof, using an iontophoresis device, the drug solution holding portion of the device comprises an allergen. In certain such embodiments, the allergen is selected from insect venoms; plant pollens; house dust mites; animal dander; ragweed; or endotoxin.

A method for administering an immune response-enhancing agent, or composition thereof, using an iontophoresis device, the device, comprising: an active electrode assembly having a drug solution holding portion, comprising an immune response-enhancing agent, or composition thereof; and a non-active electrode assembly; wherein the active electrode assembly further comprises: a first electrode member operable to provide an electrical potential of a first polarity; the drug solution holding portion arranged on the front surface of the electrode member; and a first ion-exchange membrane arranged on the front surface of the drug solution holding portion; and wherein the non-active electrode assembly comprises: a second electrode member operable to provide an electrical potential of a second polarity; and a first electrolyte solution holding portion arranged on the front surface of the second electrode member; the method comprising: electrically coupling the active electrode assembly and the non-active electrode assembly to poles of a power source; and applying a voltage or current to the active electrode assembly and the non-active electrode assembly; wherein the active electrode assembly and the non-active electrode assembly are brought into contact with a skin of a mammal.

In certain embodiments of the method for administering an immune response-enhancing agent, or composition thereof, using an iontophoresis device, the active electrode assembly of the device further comprises: a second electrolyte solution holding portion arranged on the front surface of the first electrode member; and a second ion-exchange membrane interposed between the second electrolyte solution holding portion and the drug solution holding portion. In certain other embodiments of the method, the non-active electrode assembly of the device further comprises: a third ion-exchange membrane arranged on the front surface of the first electrolyte solution holding portion. In certain other embodiments of the method, the non-active electrode assembly further comprises: a fourth ion-exchange membrane arranged on the front surface of the first electrolyte solution holding portion; and a third electrolyte solution holding portion interposed between the fourth ion-exchange membrane and the third ion-exchange membrane. In certain other embodiments of the method, the first polarity of the device is a negative polarity; the second polarity of the device is a positive polarity; the first ion-exchange membrane and the fourth ion-exchange membrane of the device are anion-exchange membranes; the second ion-exchange membrane and the third ion-exchange membrane of the device are cation-exchange membranes; and the immune response-enhancing agent is lipid A or a lipid A analogue. In yet other embodiments of the method, the lipid A analogue is selected from monophosphoryl lipid A (MPL); 3-O-deacylated monophosphoryl lipid A; and aminoalkylglucosaminide 4-phosphate.

In various embodiments, an iontophoresis device and a method are provided for administration of any of a variety of adjuvants, including lipid A and lipid A analogues to a mammal in such a manner that immune response-enhancing or immune response-stimulating effects can be produced effectively, safely, and painlessly.

In certain other embodiments, there are provided an iontophoresis device and a method capable of administering adjuvants, such as lipid A or lipid A analogues, to a living organism in such a manner that immune response-enhancing or immune response-stimulating effects can be sufficiently produced under current application conditions under which no damage, no pain, or no stimulation exceeding an allowable limit is given to the skin of the living organism, or in such a manner that immune response-enhancing or immune response-stimulating effects equivalent to or greater than those of intracutaneous injection can be produced.

In certain other embodiments, there are provided an iontophoresis device and a method capable of administering lipid A or lipid A analogues to a living organism in such a manner that immune response-enhancing or immune response-stimulating effects can be sufficiently produced in an administration time that is acceptable as a time for administering a drug or agent, or in such a manner that immune response-enhancing or immune response-stimulating effects equivalent to or more than those of intracutaneous injection can be produced.

### BRIEF DESCRIPTION OF THE DRAWINGS

In the accompanying drawings:

FIG. 1 is a schematic view showing a configuration of an iontophoresis device according to an embodiment of the present invention.

FIG. 2 is a schematic view showing a configuration of an iontophoresis device according to another embodiment of the present invention.

FIG. 3 is a schematic view showing a configuration of an iontophoresis device according to still another embodiment of the present invention.

FIG. 4 is a schematic view showing a configuration of an iontophoresis device used in an MPL administration experiment.

FIG. 5(1) and 5(2) are graphs showing IgG1 and IgG2 antibody titers on day 43.

### DETAILED DESCRIPTION OF THE INVENTION

In the following description, certain specific details are set forth in order to provide a thorough understanding of various disclosed embodiments. However, one skilled in the relevant art will recognize that embodiments may be practiced without one or more of the these specific details, or with other methods, components, materials, etc. In other instances, well-known structures associated with controllers including but not limited to voltage and/or current regulators have not been shown or described in detail to avoid unnecessarily obscuring descriptions of the embodiments.

Unless the context requires otherwise, throughout the specification and claims which follow, the word “comprise” and variations thereof, such as, “comprises” and “comprising” are to be construed in an open, inclusive sense, that is, as “including, but not limited to.”

Reference throughout this specification to “one embodiment” or “an embodiment” means that a particular feature, structure, characteristic or aspect of a method described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, characteristics, or aspects of a method may be combined in any suitable manner in one or more embodiments.

As used herein and in the claims, an “active electrode assembly” is an electrode assembly holding drugs or active agents. A “non-active electrode assembly” is an electrode assembly that functions as a counter electrode to the active electrode assembly.

As used herein and in the claims, the term “membrane” means a layer, barrier or material, which may or may not be permeable. Unless specified otherwise, membranes may take the form of a solid, liquid or gel, and may or may not have a distinct lattice or cross-linked structure. An “anion-exchange membrane” refers to a membrane having functional groups that enable it to bind and release negatively charged ions. An anion-exchange membrane in an iontophoretic device permits the passage only of anions and substantially blocks the passage of cations. A “cation-exchange membrane” refers to a membrane having functional groups that enable it to bind and release positively charged ions. A cation-exchange membrane in an iontophoretic device permits the passage only of cations and substantially blocks the passage of anions.

As used herein and in the claims, the term “skin” refers to the organism surface or biological interface, including mucous membranes, at which delivery of a drug or active agent can be carried out by iontophoresis.

As used herein and in the claims, the term “drug” or “active agent” refers to an agent, a substance, or a compound that elicits some type of action or biological response when delivered to a mammal, including a human. A “drug” or “active agent” can be an immunological agent, an adjuvant, an immune response-enhancing agent, a vaccine, an antigen, a drug, a hormone, a protein, a peptide, or a nucleic acid such as DNA. Many biologically active agents have functional groups that may be converted to a charged ion or may dissociate into a charged ion and a counter ion in an aqueous medium at an appropriate pH. Other drugs or active agents may be polarized or polarizable, that is exhibiting a polarity at one portion relative to another portion of the molecule.

The headings provided herein are for convenience only and do not interpret the scope or meaning of the embodiments.

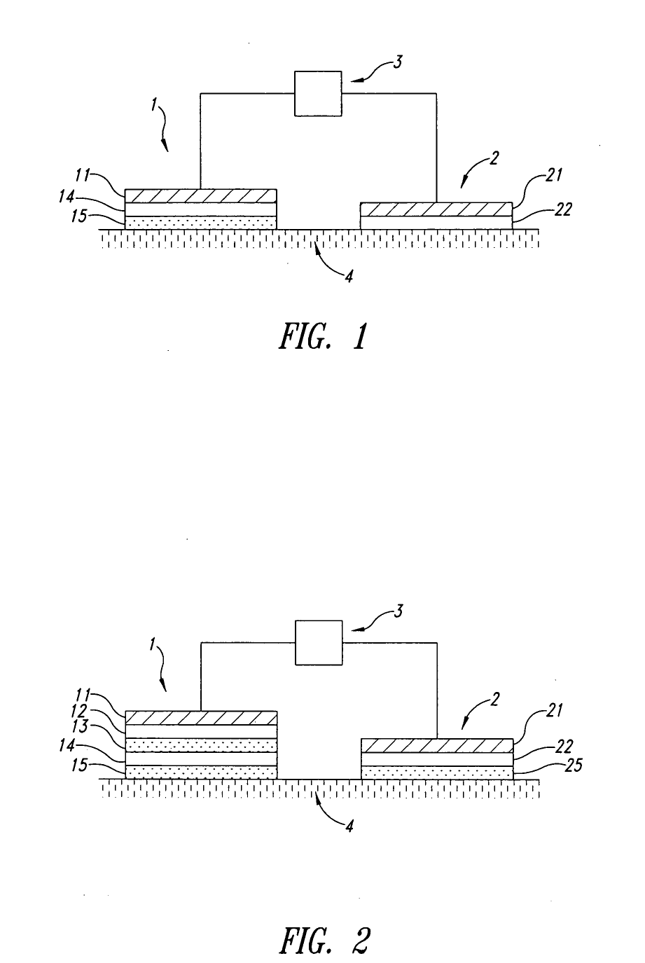

FIGS. 1 to 3 are each a schematic cross-section showing a basic structure of an iontophoresis device.

The device includes as major constituent elements (members) an active electrode assembly 1, and a non-active electrode assembly 2, electrically coupled to a power source 3, operable to supply one or more drugs or active agents contained in the active electrode assembly 1 to a site of skin (or mucous membrane) 4.

In the embodiment shown in FIG. 1, the active electrode assembly 1 comprises an electrode member 11 operable to provide an electrical potential of a first polarity; a drug solution holding portion 14 arranged at the front surface of the electrode member 11; and an ion exchange membrane 15 arranged on the front surface of the drug solution holding portion 14. The non-active electrode assembly 2 comprises an electrode member 21 operable to provide an electrical potential of a second polarity; and an electrolyte solution holding portion 22 arranged on the front surface of the electrode member 21.

In one embodiment of the device shown in FIG. 1, the electrode element 11 of the active electrode assembly 1 is electrically coupled to a negative pole of the power source 3; the electrode element 21 of the active electrode assembly 2 is electrically coupled to a positive pole of the power source 3; and the ion-exchange membrane.

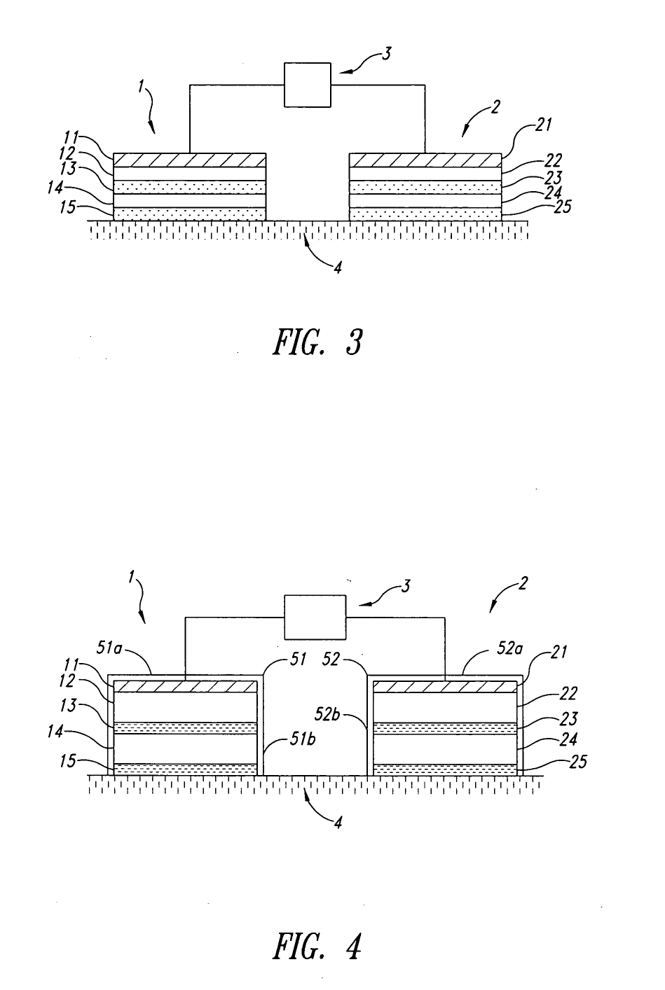

In each of the embodiments shown in FIGS. 2 and 3, the active electrode assembly 1 comprises an electrode member 11 operable to provide an electrical potential of a first polarity; an electrolyte solution holding portion 12 arranged on the front surface of electrode member 11; an ion-exchange membrane 13 arranged on the front surface of the electrolyte solution holding portion 12; a drug solution holding portion 14 arranged at the front surface of the ion-exchange membrane 13; and an ion exchange membrane 15 arranged on the front surface of the drug solution holding portion 14.

In the embodiment shown in FIG. 2, the non-active electrode assembly 2 comprises an electrode member 21 operable to provide an electrical potential of a second polarity; an electrolyte solution holding portion 22 arranged on the front surface of the electrode member 21; and an ion-exchange membrane 23 arranged on the front surface of the electrolyte solution holding portion 22.

In the embodiment shown in FIG. 3, the non-active electrode assembly 2 comprises an electrode member 21 operable to provide an electrical potential of a second polarity; an electrolyte solution holding portion 22 arranged on the front surface of the electrode member 21; an ion-exchange membrane 23 arranged on the front surface of the electrolyte solution holding portion 22; an electrolyte solution holding portion 24 arranged on the front surface of the ion-exchange membrane 23; and an ion-exchange membrane 25 arranged on the front surface of the electrolyte solution holding portion 24.

In certain embodiments, the working or active electrode member 11 and the nonworking or counter electrode member 21 may be preferably electrochemically inactive electrodes made of carbon, platinum and so on. It is particularly preferable that these carbon electrodes may advantageously ensure that metal ions are not eluted and do not migrated into the living organism.

However, it is also possible to adopt an electrochemically active electrode, for example, a silver/silver chloride couple electrode that includes the working or active electrode member 11 made of silver chloride and the nonworking or counter electrode member 21 made of silver.

For example, assume a silver/silver chloride coupled electrode is used. On the nonworking or counter electrode, which is an anode (positive electrode) in the case of the device for delivering lipid A or analogues thereof, silver electrode and chloride ion (Cl⁻) readily react to form water-insoluble AgCl by the reaction: Ag⁺ Cl⁻→AgCl+e⁻. On the working or active electrode, which is a cathode (negative electrode) in this case, a reaction in which chloride ion (Cl⁻) elutes from the silver chloride electrode occurs. As a result, electrolysis reaction of water is prevented, so that acidification due to H⁺ ion on the anode (positive electrode) and alkalation due to OH⁻ ion on the cathode (negative electrode) can be prevented.

In contrast, in the active electrode assembly 1 and the non-active electrode assembly 2 in the iontophoresis devices shown in FIGS. 2 and 3, the alkalation due to OH⁻ ion in the electrolyte solution holding portion 12 and the acidification due to H⁺ ion in the electrolyte solution holding portion 22 may be prevented by the action of anion-exchange membrane and/or cation-exchange membrane. Accordingly, in the iontophoresis devices shown in FIGS. 1 to 3, particularly the iontophoresis devices shown in FIGS. 2 and 3, carbon electrodes that are inexpensive and free of the concern over elution of metal ions can be used advantageously instead of active electrodes such as the silver/silver chloride couple electrode.

The electrolyte solution holding portion 12, 22, and 24 in the iontophoresis devices hold electrolytes for securing electrical conductivity. Typical examples of the electrolytes that can be used include phosphate buffered saline and physiological saline.

The electrolyte solution holding portion 12 and 22 can contain a compound that is oxidized or reduced more easily than electrolysis reaction of water (oxidation on the positive electrode and reduction on the negative electrode) in order to effectively prevent generation of gas and a change in pH by the electrolysis of water. From the viewpoint of biocompatibility with living organisms and economy (being inexpensive and easily available), it may be preferable to use, for example, inorganic compounds such as ferrous sulfate and ferric sulfate, medical agents such as ascorbic acid (vitamin C) and sodium ascorbate, acidic compounds that are present on the surface of the skin, such as lactic acid, organic acids such as oxalic acid, malic acid, succinic acid, and fumaric acid and/or salts thereof. These can be used singly or in combinations.

That is, in the electrolyte solution holding portion 12 and 22, electrochemical reaction occurs to decompose the electrolyte or decompose the ionic drug. As a result, bubbles may be generated in the electrolyte solution holding portion 12 and 22 to prevent the electrode materials 11 and 21 from contacting the electrolyte. For example, hydrogen gas may be generated on the negative electrode. Chlorine gas and oxygen gas may be generated on the positive electrode. In this situation, resistance increases due to the bubbles and current does not flow even when the voltage is increased further. This may be a very serious problem from the viewpoint of the practical utility of the iontophoresis device.

Such a cause of instability may be be eliminated by addition of the above-mentioned compounds, for example, by using a 1:1 mixed aqueous solution of 1 molar (M) lactic acid and 1 molar (M) sodium fumarate.

To prevent changes in composition of the electrolyte solution holding portion 12 and the drug solution holding portion 14 that is explained below on due to mixing of the electrolyte solution holding portion 12 with the drug solution holding portion 14 (having, for example, an aqueous solution of lipid A or lipid A analogues), the electrolyte solution holding portion 12 can contain the same material as that in the drug solution holding portion 14 (for example, aqueous solution of lipid A or lipid A analogues). In the case of the electrolyte solution holding portion 24, the compositions of the electrolyte solution holding portions 22 and 24 can be similar or the same to prevent a change in composition of the electrolyte solution holding portion 24 due to mixing with the medium in the electrolyte solution holding portion 22.

The electrolyte solution holding portion 12, 22, and 24 may contain the above-described electrolyte in a liquid state. However, it is also possible to impregnate a water-absorbing thin film made of a polymer material with the above-mentioned electrolyteto increase their handleability. The film used herein can be the same as that can be used in the drug solution holding portion 14 and details of the film will be explained later on when the drug solution holding portion 14 is explained.

Suitable cation-exchange membranes may include NEOSEPTAs (CM-1, CM-2, CMX, CMS, CMB, CLE04-2 and so on) manufactured by Tokuyama Co., Ltd., Tokyo, Japan. Suitable anion-exchange membranes may include NEOSEPTAs (AM-1, AM-3, AMX, AHA, ACH, ACS, ALE04-2, AIP-21 and so on) manufactured by Tokuyama Co., Ltd. Among them, a cation-exchange membrane that includes a porous film having cavities in a portion or whole of which cavities an ion-exchange resin having a cation-exchange function is filled, or an anion-exchange membrane that includes a porous film having cavities in a portion or whole of which cavities an ion-exchange resin having an anion-exchange function is filled may be preferable in some applications.

The above-mentioned ion-exchange resins can be fluorine-based ones that include a perfluorocarbon skeleton having an ion-exchange group and hydrocarbon-based ones that include nonfluorinated resin as a skeleton. From the viewpoint of convenience of production process, hydrocarbon-based ion-exchange resins may be preferable. The filling rate of the ion-exchange resin depends on the porosity of the porous film and generally is 5 to 95 mass %, or 10 to 90 mass %, or 20 to 60 mass %.

The ion-exchange group in the above-mentioned ion-exchange resin is not particularly limited so far as it is a functional group that generates a group having a negative or positive charge in aqueous solutions. Specific examples of the functional group that can serve as such an ion-exchange group include cation exchange groups such as a sulfonic acid group, a carboxylic acid group, and a phosphonic acid group. These acid groups can be present as free acids or in the form of salts. Counter cations for the salts of the acids include alkali metal ions such as sodium ion and potassium ion, and ammonium ion. Among these cation-exchange groups, generally, a sulfonic acid group, which is a strong acid group, is may be particularly preferable. The anion-exchange groups include, for example, a primary amino group, a secondary amino group, a tertiary amino group, a quaternary ammonium group, a pyridyl group, an imidazole group, a quaternary pyridinium group, and a quaternary imidazolium group. Counter anions for these anion-exchange groups include halogen ions such as chlorine ion, hydroxy ion, and so on. Among these anion-exchange groups, generally a quaternary ammonium group and a quaternary pyridinium group, which are strong basic groups, may be preferable.

The above-mentioned porous film is not particularly limited and any porous film can be used as far as it is in the form of a film or a sheet that has a lot of pores communicating both sides thereof. To satisfy both of high strength and flexibility, it is preferable that the porous film be made of a thermoplastic resin.

Examples of the thermoplastic resins constituting the porous film include, without limitation: polyolefin resins such as homopolymers or copolymers of α-olefins such as ethylene, propylene, 1-butene, 1-pentene, 1-hexene, 3-methyl-1-butene, 4-methyl-1-pentene, and 5-methyl-1-heptene; vinyl chloride resins such as polyvinyl chloride, vinyl chloride-vinyl acetate copolymers, vinyl chloride-vinylidene chloride copolymers, and vinyl chloride-olefin copolymers; fluorine resins such as polytetrafluoroethylene, polychlorotrifluoroethylene, polyvinylidene fluoride, tetrafluoroethylene-hexafluoropropylene copolymers, tetrafluoroethylene-perfluoroalkyl vinylether copolymers, and tetrafluoroethylene-ethylene copolymers; polyamide resins such as nylon 6 and nylon 66; and those which are made from polyamide resins. Polyolefin resins may be preferred as they are superior in mechanical strength, flexibility, chemical stability, and chemical resistance, and have good compatibility with ion-exchange resins. As the polyolefin resins, polyethylene and polypropylene may be particularly preferable and polyethylene may be most preferable, depending on the specific application.

The physical properties of the above-mentioned porous film made of the thermoplastic resin are not particularly limited. However, it may be preferable that the pore has a mean pore size of preferably 0.005 μm to 5.0 μm, or may more preferably be 0.01 μm to 2.0 μm, or may most preferably be 0.02 μm to 0.2 μm because ion exchange membranes that are thin and have excellent strengths and low electric resistances can be readily obtained. The above-mentioned mean pore size as used herein means mean flow pore size measured by the bubble point method according to JIS-K3832-1990. A porosity of the porous film of 20 to 95% may be preferred, while 30 to 90% may be more preferred, and 30 to 60% may be most preferred, depending on the application. To obtain ion-exchange membranes that have a thickness as described below, the thickness of the porous film of 5 μm to 140 μm may be preferred, while 10 μm to 120 μm may be more preferred, and 15 μm to 55 μm may be most preferred, depending on the specific application. Usually, anion-exchange membranes and cation-exchange membranes that include such porous films have the same thickness as that of the porous film or up to about 20 μm larger than the thickness of the porous film.

The drug solution holding portion 14 in the iontophoresis device of the present invention holds an aqueous solution that contains at least one of lipid A or lipid A analogues (as exemplifying any of a variety of adjuvants). Because the lipid A or lipid A analogues dissociate into negatively-charged ion when dissolved in water, the resultant aqueous solution contains negatively-charged ion of the lipid A or lipid A analogues.

The drug solution holding portion 14 can be configured to hold the aqueous solution of the lipid A or lipid A analogues in a liquid state. When the aqueous solution of the lipid A or lipid A analogues is impregnated in and held by the following water-absorbing thin film, the handleability and other properties of the drug solution holding portion 14 can increase.

Examples of the material that can be used as the water-absorbing thin film as described above include hydrogel forms of acrylic resins (acrylic hydrogel film), a segmented polyurethane-based gel film, and an ion-conductive porous sheet for forming a gel-like solid electrolyte. When the film is impregnated with the above-mentioned aqueous solution is impregnated at an impregnation rate of 30% to 40%, a high transport number (high drug delivery), for example, 70% to 80% can be obtained.

The impregnation rate as used herein is by % by weight and is defined by 100×(W−D)/D (%) wherein D indicates dry weight and W indicates weight after impregnation. The impregnation rate must be measured immediately after the impregnation with the aqueous solution to exclude influences with time.

The transport number as used herein is a ratio of current due to the migration of the medicine ion (ion of lipid A or lipid A analogues) to total current that flows through the electrolyte solution. The transport number is measured by placing the thin film impregnated with the ionic medicine between the ion-exchange membranes 13 and 15 and then assembling other component members and in such a manner that changes with time can be minimized.

The above-mentioned acrylic hydrogel film (available from, for example, Sun Contact Lens Co., Ltd.) is a gel that has a three-dimensional network (crosslinked structure). Such a gel to which an aqueous electrolyte solution as a dispersant is added serves as a polymer adsorbent with ion conductivity. The relationship between the impregnation rate and transport number of the acrylic hydrogel film can be adjusted depending on the size of the three-dimensional network as well as the kinds of and the ratios of the monomers that constitute the resin. The above-mentioned acrylic hydrogel film that has an impregnation rate of 30% to 40% and a transport number of 70% and 80% can be prepared from 2-hydroxyethyl methacrylate and ethylene glycol dimethacrylate (monomer ratio: (98 to 99.5):(0.5 to 2). It has been confirmed that within the range of 0.1 to 1 mm, which is an ordinary thickness range, the above-mentioned impregnation rate and transport number are almost the same.

The segmented polyurethane-based gel film has a segment of polyethylene glycol (PEG) and a segment of polypropylene glycol (PPG). The physical properties of the segmented polyurethane-based gel film can be adjusted by changing the ratio of the monomer that constitutes the segmented polyurethane-based gel film and diisocyanate. The segmented polyurethane-based gel film has a three-dimensional structure crosslinked through urethane bonds. Accordingly, the impregnation rate, transport number, and adhesive force can be readily adjusted by controlling the size of the three-dimensional network as well as the kinds of and the ratios of the monomers that constitute the resin in the same manner as the above-mentioned acrylic hydrogel film. In the segmented polyurethane-based gel film (porous gel film) to which water as a dispersant and an electrolyte (for example, alkali metal salt), oxygen in the ether bond of polyether that constitutes the segment and the alkali metal salt form a complex. When electricity is applied to the complex, ion of the metal salt migrates to the oxygen at the next vacant ether bond to develop electrical conductivity. The segmented polyurethane-based gel film contains a PEG-PPG-PEG copolymer that constitutes the segment. The PEG-PPG-PEG copolymer is granted for use as a cosmetic material. This indicates that the segmented polyurethane-based gel film appears to cause no irritation in the skin and is highly safe.

The ion-conductive porous sheet for forming a gel-like solid electrolyte includes, for example, one that is disclosed in JP 11-273452 A. This includes acrylonitrile copolymer as a base, and specifically porous polymer having a porosity of 20% to 80% as a base. More specifically, the above-mentioned base is an acrylonitrile copolymer that contains 50 mol % (70 mol % to 98 mol % may be preferred) or more of acrylonitrile and has a porosity of 20% to 80%. The above-mentioned acrylonitrile-based gel-like solid electrolyte sheet (solid battery) is prepared by impregnating an acrylonitrile-based copolymer sheet that is soluble in a nonaqueous solvent and has a porosity of 20% to 80% with the nonaqueous solvent containing an electrolyte and gelling the resultant. The obtained gel forms include gel-like ones to hard film-like ones.

In terms of the ion conductivity, biocompatibility, and the like, the acrylonitrile copolymer sheet soluble in a non-aqueous solvent may be composed of an acrylonitrile/C1 to C4 alkyl (meth)acrylate copolymer, an acrylonitrile/vinylacetate copolymer, an acrylonitrile/styrene copolymer, an acrylonitrile/vinylidene chloride copolymer, or the like. The copolymer sheet is made porous by an ordinary method such as a wet (dry) paper making method, a needlepunching method that is a kind of a non-woven fabric producing method, a water-jet method, drawing perforation of a melt-extruded sheet, or perforation by solvent extraction. Among the ion-conductive porous sheets made of the acrylonitrile-based copolymer used in the above-described solid battery, the gel forms (gel-like ones to hard film-like ones) that hold the above-mentioned aqueous solution in the three-dimensional network of the polymer chain are useful as thin films for use in the drug solution holding portion 14, or electrolyte solution holding portions 12, 22, and 24.

The conditions under which the above-mentioned thin film (porous gel film) is impregnated with the aqueous solution of lipid A or the aqueous solution of the lipid A analogue, or the electrically-conductive medium can be determined optimally depending on the impregnation amount, impregnation speed and so on. For example, impregnation conditions of 40° C. for 30 minutes can be selected.

The power source 3 in the iontophoresis device that can be used include, for example, a battery, a constant voltage device, a constant current device (a Galvanic device), and a constant voltage-constant current device. It may be preferable to use a constant current device whose current can be controlled within the range of 0.01 mA to 1.0 mA, although 0.01 mA to 0.5 mA may be more preferred, and that operates at safe voltage conditions, specifically, at 50 V or less, while 30 V or less may be more preferred.

Moreover, the power source 3 may be one that is capable of applying current while changing current with time.

Adjuvants generally are agents that are used to enhance the effectiveness of, for example, a pharmacological compound. In particular, adjuvants are administered with vaccines or antigens to enhance the immune response to the vaccine or antigen. In certain embodiments, any of a variety of adjuvants, herein exemplified by lipid A and lipid A analogues, may be used in the with the iontophoretic devices and methods of use thereof disclosed herein.

Lipid A is a glycolipid having a chemical structure represented by the structural formula 1 obtained from gram-negative bacteria, for example, Escherichia coli. The lipid A analogues are derivatives of lipid A. The derivatives have a disaccharide structure (4-O-2-amino-2-deoxy-β-D-glucopyranosyl-amino-2-deoxy-D-glucopyranose) consisting of two D-glucosamine molecules connected through a β1-6 bond as a basic skeleton. Examples of the lipid A analogues include monophosphoryl lipid A having a chemical structure represented by the structural formula 2 (for example, “MPL”, prepared by Corixa Corporation (Seattle, Wash., U.S.A.), 3-O-deacylated monophosphoryl lipid A disclosed in U.S. Pat. No. 4,912,094 B, and aminoalkylglucosaminide 4-phosphates having a chemical structure represented by the structural formula 3 (for example, “RC-529”, manufactured by Corixa Corporation supra). MPL can be isolated and prepared from natural sources or synthetic preparations may be obtained MPL

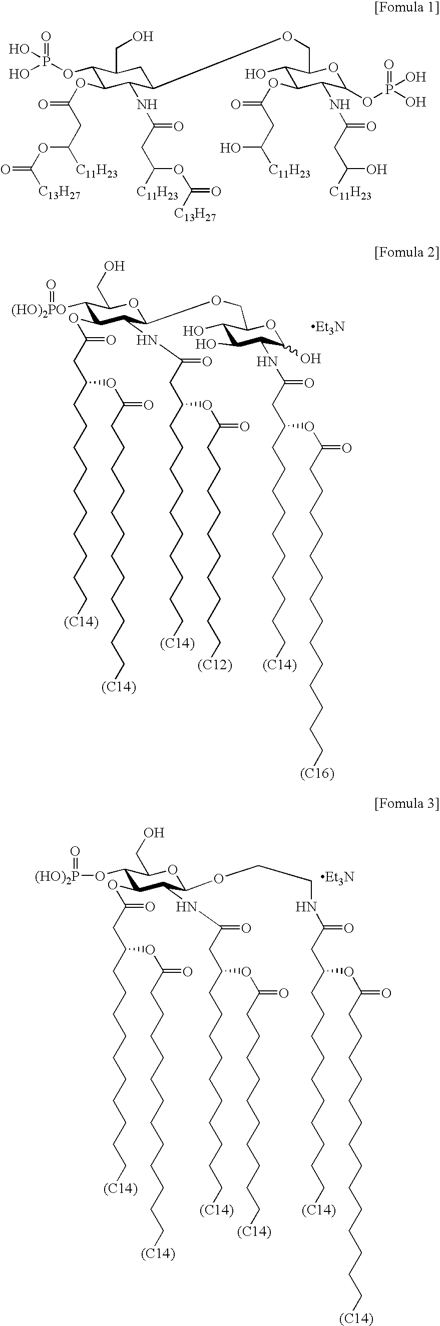

The iontophoresis device and the method of administering lipid A or lipid A analogues can be used and practiced, respectively, in combination with administration of vaccines and allergens into a living organism by injection. For example, the iontophoresis device can be used to administer lipid A or lipid A analogues into a living organism simultaneously with or after a predetermined time from the injection of the vaccine or the allergen. This can lead to an increase in the effects of the vaccine or the allergen.

The lipid A or lipid A analogues contained in the drug solution holding portion can be agonists of Toll-like receptors (TLR), examples of which include TLR-2, TLR-4, TLR-5, TLR-7, and/or TLR-9.

The drug solution holding portion can be configured to contain vaccine or allergen in addition to lipid A or lipid A analogues. With this configuration, lipid A or lipid A analogues can be transcutaneously administered simultaneously with the vaccine or the allergen.

Examples of such vaccines that can be used include hepatitis antigen, type B hepatitis surface antigen, type B hepatitis surface antigen mutant, influenza antigen, leishmaniasis antigen and endotoxin. Alternatively, one or more of substances obtained from non-hepatitis antigen that have protective effects on one or more of pathogenic microbes or virus such as Bordetella pertussis, Corynebacterium diphtheriae, Chlostridium tetani, pertussis, influenza B virus, or polio virus; mixtures of DTP (diphtheria, tetanus, and pertussis) and HBsAg (type B hepatitis surface antigen), mixtures of Hib (influenza B virus) and HBsAg, mixtures of DTP, HBsAg, and Hib, or mixtures of IPV (inactivated polio vaccine), DTP, HBsAg, and Hib, and so on can be used.

In other embodiments, the iontophoresis device and the method of administering lipid A or lipid A analogues can be configured to contain, in addition to, or instead of, lipid A or lipid A analogues, one or more adjuvants, such as other agonists of toll-like receptors (such as TLR-2, TLR-4, TLR-5, TLR-7, and TLR-9); saponin, such as QS-21, or derivatives thereof; or CpG (as disclosed in U.S. Pat. No. 5,856,462 B, the contents of which are incorporated herein by reference).

In certain other embodiments, the iontophoresis device and the method of administering lipid A or lipid A analogues can be configured to contain, in addition to, or instead of, lipid A or lipid A analogues, imiquimod; resiquimod; or dSLIM.

The iontophoresis device and the method of administering lipid A or lipid A analogues can be configured to contain, in addition to lipid A or lipid A analogues, imiquimod or flagellin.

The iontophoresis device and the method of administering lipid A or lipid A analogues can be configured such that the above-mentioned drug solution holding portion contains in addition to lipid A or lipid A analogues, one or more of allergens such as pollens, mites, which constitutes house dust, dander (minute dropouts from feather, skin, hair and so on of animals), and ragweed (Ambrosia artemisiaefolia var. elatior). With this configuration, the iontophoresis device and the method of administering lipid A or lipid A analogues can be used or practiced in the therapy of allergic diseases.

### EXAMPLE

The following experiments were carried out to evaluate immune response-enhancing effects obtained when the lipid A analogue monophosphoryl lipid A (MPL) is administered using an iontophoresis device.

Vaccine

A vaccine for tuberculosis (Mtb72F, obtained from Corixa Corporation, Seattle, Wash.) was used.

Adjuvant

MPL, a clinical test preparation of monophosphoryl lipid A produced by Corixa, was used as an adjuvant. As reference data, MPL-AF, a hydrophilic preparation of monophosphoryl lipid A prepared by Corixa and MPL-SE, a lipophilic preparation of monophosphoryl lipid A prepared by Corixa were intracutaneously injected and their immunostimulating effects were evaluated.

Test Animals

57BL/6 mice (7 to 24 weeks, female) were used.

Experimental Conditions

The above-mentioned 57BL/6 mice were divided into four groups each consisting of 2 to 5 mice. To the animals in each group were administered vaccine (Mtb72F) and an adjuvant (MPL, MPL-AF, or MPL-SE).

The administration schedules for vaccine and adjuvant to each group were as described in “Contents of experiment” below.

### Group 1: (Example)

(a) Mtb72F: Administered by intracutaneous injection

(b) MPL: Administered transcutaneously using a TCT apparatus (the apparatus described in “Apparatus used”);

### Group 2: (Comparative Example 1)

(a) Mtb72F: Administered by intracutaneous injection

(b) MPL-AF: Administered by intracutaneous injection;

### Group 3: (Comparative Example 2)

(a) Mtb72F: Administered by intracutaneous injection

(b) MPL-SE: Administered by intracutaneous injection;

### Group 4: (Comparative Example 3)

(a) Mtb72F: Administered by intracutaneous injection

(b) MPL-AF: Not administered.

Apparatus Used

For administering MPL to Group 1 mice (Example), the iontophoresis device shown in FIG. 4 was used.

In FIG. 4, the apparatus includes an active electrode assembly 1, a non-active electrode assembly 2, and a constant current power source 3.

The active electrode assembly 1 includes a cylindrical acrylic vessel 51, which has a top wall 51a and a side wall 51b and is open at the lower end. In the vessel 51, a carbon electrode element 11, having a diameter of about 10 mm and connected to the negative electrode of the constant current source 3, a cation-exchange membrane 13 (CLE04, manufactured by Tokuyama Co., Ltd., Tokyo, Japan), and an anion-exchange membrane 15 (AIP-21 manufactured by Tokuyama Co., Ltd.) are arranged in the order shown in FIG. 4.

A space between the carbon electrode 11 and the cation-exchange membrane 13 constitutes an electrolyte solution holding portion 12 that contains about 0.8 ml of an electrically-conductive medium in a liquid state. A space between the cation-exchange membrane 13 and the anion-exchange membrane 15 constitutes a drug solution holding portion 14 that contains about 1.2 ml of a drug in a liquid state.

In this example, an aqueous MPL solution having dissolved 300 μg of MPL in 15 ml of sterilized water was injected into the drug solution holding portion 14 as a drug solution. An aqueous MPL solution that has the same composition as the above-mentioned drug solution was used as an electrically-conductive medium for the electrolyte solution holding portion 12.

The non-active electrode assembly 2 includes a cylindrical acrylic vessel 52, which has a top wall 52a and a side wall 52b and is open at the lower end. In the vessel 52, a carbon electrode 22, having a diameter of about 20 mm (φ) and connected to the positive electrode of the constant current source 3, an anion-exchange membrane 23 (ALE04-2, manufactured by Tokuyama Co., Ltd.), and a cation-exchange membrane 25 (CLE04-2 manufactured by Tokuyama Co., Ltd.) were arranged in the order shown in FIG. 4.

A space between the carbon electrode 21 and the anion-exchange membrane 23 and a space between the cation-exchange membrane 23 and the anion-exchange membrane 25 constitute electrolyte solution holding portions 22 and 24, respectively, that contain about 0.8 ml and about 1.2 ml, respectively, of electrically-conductive medium in a liquid state.

In this example, phosphate-buffered saline was used as the electrically-conductive medium in the electrolyte solution holding portions 22 and 24.

Galvanostat (HA5010m, manufactured by Hokuto Denko Co., Ltd., Tokyo, Japan) was used as the constant current power source 3.

Contents of Experiment

Day 1:

(A) 10 μg of the vaccine (Mtb72F) was injected into the base of the tail of each mouse in Groups 1 to 4.

(B) Subsequently, the adjuvants (MPL, MPL-AF, or MPL-SE) were administered to mice in Groups 1 to 4 by the following method.

Group 1: transcutaneous administration of MPL was performed using the above-mentioned TCT apparatus.

The target mice were subjected to depilation treatment (shaving after being coated with depilatory cream) the previous day, and the active electrode assembly 1 and the non-active electrode assembly 2 of the iontophoresis device were abutted to the abdomen of the mouse with an adhesive. Current was applied under the following conditions for 30 minutes.

| | |
|---|---|
| 0 to 15 minutes | 0.02 mA |
| 15 to 27 minutes | 0.04 mA |
| 27 to 30 minutes | 0.15 mA |

Group 2: MPL-AF (20 μg) was intracutaneously injected at a site 1 inch (2.54 μm) from the base of the tail of the mouse.

Group 3: MPL-SE (20 μg) was intracutaneously injected at a site 1 inch (2.54 cm) from the base of the tail of the mouse.

Group 4: No treatment

Day 22: Blood samples were collected from the mice and antibody (IgG1 and IgG2a) reaction was tested by a conventional method.

Day 35: A boost (additional immunizing) was performed on the mice in Groups 1 to 4 in the same manner as that on Day 1.

Day 43: Blood samples were collected from the mice and antibody (IgG1 and IgG2a) reaction was tested by a conventional method. Further, two mice were selected from each group, and the spleens were extracted and cultivated in vitro using 10 μg/ml mtb72F, ConA (concanavalin A), PPD (tuberculin-purified protein), and a solvent to carry out irritation tests. After 72 hours, the supernatant was collected and immunogenic growth and cytokine (IFN-γ) were evaluated according to a conventional manner.

Results

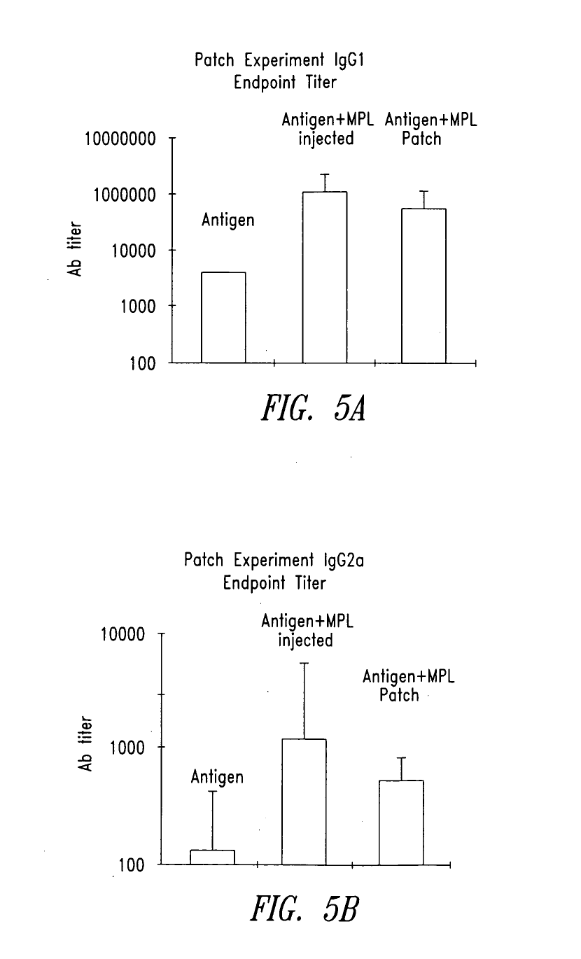

FIGS. 5(1) and 5(2) show antibody titers of IgG1 and IgG2 on day 43. In FIGS. 5(1) and 5(2), the line segments above bar graphs indicate standard deviations.

FIGS. 5(1) and 5(2) clearly demonstrate that the antibody titers generated upon transcutaneous administration of MPL, either iontophoretically or by intracutaneous injection, were significantly higher for both IgG1 and IgG2 than in the case in which no MPL was administered. The results obtain with either iontophoretic delivery or intracutaneous injection of MPL were nearly identical.

From the above, it has been confirmed that the administration of MPL iontophoretically afforded significant immune response-enhancing effects equivalent to those obtained by intracutaneous injection.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing, it will be appreciated that, although specific embodiments of the invention have been described herein for purposes of illustration, various modifications may be made without deviating from the spirit and scope of the invention. Accordingly, the invention is not limited except as by the appended claims.

1.-47. (canceled)

48. An iontophoresis device for administering an immune response-enhancing agent, or composition thereof, the iontophoresis device comprising:
an active electrode assembly having a drug solution holding portion, comprising an immune response-enhancing agent, or composition thereof;
an active electrode element operable to apply an electrical potential of a first polarity to drive at least a portion of the immune response-enhancing agent, or composition thereof, from the iontophoresis device
a non-active electrode assembly having a non-active electrode element operable to apply an electrical potential of a second polarity.

49. The device of claim 48 wherein the immune response-enhancing agent is an adjuvant.

50. The device of claim 49 wherein the adjuvant is lipid A or an analog of lipid A; an agonist of a toll-like receptor; a saponin or a derivative thereof; CpG; imiquimod; resiquimod; or dSLIM.

51. The device of claim 50 wherein the analog of lipid A is selected from monophosphoryl lipid A (MPL), 3-O-deacylated monophosphoryl lipid A, or aminoalkylglucosaminide 4-phosphate.

52. The device of claim 50 wherein the toll-like receptor is selected from TLR-2, TLR-4, TLR-5, TLR-7, or TLR-9.

53. The device of claim 50 wherein the saponin is QS-21.

54. The device of any one of claims 48 to 53 wherein the drug solution holding portion further comprises a vaccine or antigen.

55. The device of claim 54 wherein the vaccine or antigen comprises at least one antigen selected from viral antigens; bacterial antigens including bacterial endotoxin; protozoal antigens; parasite antigens; hepatitis antigens including hepatitis A, hepatitis B, and hepatitis C; hepatitis B surface antigen (HBsAg); mutants of hepatitis B surface antigen; influenza antigens; Bordetella pertussis (pertussis) antigens; Corynebacterium diphtheriae (diphtheria) antigens; Chlostridium tetani (tetanus) antigens; influenza B viral antigens; polio virus antigens; or cancer antigens.

56. The device of claim 55 wherein the parasite antigen is selected from leishmania antigens or malaria antigens.

57. The device of claim 55 wherein the cancer antigen is selected from melanoma antigens; basal cell carcinoma antigens; breast cancer antigens; prostate cancer antigens; lung cancer antigens; or ovarian cancer antigens.

58. The device of claim 54 wherein the vaccine or antigen comprises an antigen mixture selected from mixtures of DTP (diphtheria, tetanus, pertussis) and HBsAg (hepatitis B surface antigen); mixtures of Hib (haemophilus influenzae type b) and HBsAg; mixtures of DTP, HBsAg, and Hib; or mixtures of IPV (inactivated polio vaccine), DTP, HBsAg, and Hib.

59. The device of any one of claims 48 to 53 wherein the drug solution holding portion further comprises an allergen.

60. The device of claim 59 wherein the allergen is selected from insect venoms; plant pollens; house dust mites; animal dander; ragweed; or endotoxin.

61. The device of claim 48 wherein the active electrode assembly further comprises:
a first ion exchange membrane arranged on a front surface of the drug solution holding portion; and
wherein the non-active electrode assembly further comprises:
a first electrolyte solution holding portion arranged on a front surface of the non-active electrode element.

62. The device of claim 61 wherein the active electrode assembly further comprises:
a second electrolyte solution holding portion arranged on a front surface of the active electrode element; and
a second ion exchange membrane interposed between the second electrolyte solution holding portion and the drug solution holding portion.

63. The device of claim 62 wherein the non-active electrode assembly further comprises:
a third ion exchange membrane arranged on a front surface of the first electrolyte solution holding portion.

64. The device of claim 63 wherein the non-active electrode assembly further comprises:
a third electrolyte solution holding portion arranged on a front surface of the third ion exchange membrane; and
a fourth ion-exchange membrane arranged on a front surface of the third electrolyte solution holding portion.

65. The device of claim 64 wherein the first polarity is a negative polarity; the second polarity is a positive polarity; the first ion exchange membrane and the fourth ion exchange membrane are anion exchange membranes; the second ion exchange membrane and third ion exchange membrane are cation exchange membranes; and the immune response-enhancing agent is lipid A or a lipid A analog.

66. The device of claim 65 wherein the lipid A analog is selected from monophosphoryl lipid A (MPL); 3-O-deacylated monophosphoryl lipid A; or aminoalkylglucosaminide 4-phosphate.

67. A method for administering an immune response-enhancing agent, or composition thereof, using an iontophoresis device, comprising an active electrode assembly having a drug solution holding portion, comprising an immune response-enhancing agent, or composition thereof; and a non-active electrode assembly; the method comprising:
electrically coupling the active electrode assembly and the non-active assembly to poles of a power source; and
applying a voltage or current to the active electrode assembly and the non-active electrode assembly for a period of time;
wherein the active electrode assembly and the non-active electrode assembly are brought into contact with a skin of a mammal.

68. The method of claim 67 wherein the immune response-enhancing agent is an adjuvant.

69. The method of claim 68 wherein the adjuvant is lipid A or an analog of lipid A; an agonist of a toll-like receptor; a saponin or a derivative thereof; CpG; imiquimod; resiquimod; or dSLIM.

70. The method of claim 69 wherein the analog of lipid A is selected from monophosphoryl lipid A (MPL); 3-O-deacylated monophosphoryl lipid A; or aminoalkylglucosaminide 4-phosphate.

71. The method of claim 69 wherein the toll-like receptor is selected from TLR-2; TLR-4; TLR-5; TLR-7; or TLR-9.

72. The method of claim 69 wherein the saponin is QS-21.

73. The method of any one of claims 67 to 72 wherein the drug solution holding portion further comprises a vaccine or antigen.

74. The method of claim 73 wherein the vaccine or antigen comprises at least one antigen selected from viral antigens; bacterial antigens including bacterial endotoxin; protozoal antigens; parasite antigens; hepatitis antigens including hepatitis A, hepatitis B, and hepatitis C; hepatitis B surface antigen (HBsAg); mutants of hepatitis B surface antigen; influenza antigens; Bordetella pertussis (pertussis) antigens; Corynebacterium diphtheriae (diphtheria) antigens; Chlostridium tetani (tetanus) antigens; influenza B viral antigens; polio virus antigens; or cancer antigens.

75. The method of claim 74 wherein the parasite antigen is selected from leishmania antigens or malaria antigens.

76. The method of claim 74 wherein the cancer antigen is selected from melanoma antigens; basal cell carcinoma antigens; breast cancer antigens; prostate cancer antigens; lung cancer antigens; or ovarian cancer antigens.

77. The method of claim 73 wherein the vaccine or antigen comprises an antigen mixture selected from mixtures of DTP (diphtheria, tetanus, pertussis) and HBsAg (hepatitis B surface antigen); mixtures of Hib (haemophilus influenzae type b) and HBsAg; mixtures of DTP, HBsAg, and Hib; or mixtures of IPV (inactivated polio vaccine), DTP, HBsAg, and Hib.

78. The method of any one of claims 67 to 72 wherein the drug solution holding portion further comprises an allergen.

79. The method of claim 78 wherein the allergen is selected from insect venoms; plant pollens; house dust mites; animal dander; ragweed; or endotoxin.
